(19) United States
(12) Patent Application Publication
(10) Pub. No.: US 2007/0219504 A1
(43) Pub. Date: Sep. 20, 2007

(54) Needle protection device with gauge specific color coding and method for manufacturing thereof

(75) Inventors: David MacLean, Swanzey, NH (US); Frank Blinkhorn, Keene, NH (US); Troy M. Derby, Stoddard, NH (US); Quinton Farrar, Keene, NH (US); Marius Hauri, Westmoreland, NH (US); Lawrence P. Hudon, Hinsdale, NH (US); Robert Simas JR., Keene, NH (US)

Correspondence Address: LOUIS WOO;LAW OFFICE OF LOUIS WOO, 717 NORTH FAYETTE STREET, ALEXANDRIA, VA, 22314, US

(73) Assignee: Smiths Medical ASD, Inc., Keene, NH (US)

(21) Appl. No.: 11/785,504

(22) Filed: Apr. 18, 2007

Related U.S. Application Data
(62) Division of application No. 10/751,982, filed on Jan. 7, 2004.

(51) Int. Cl. A61M 5/32

(52) U.S. Cl. 604/192; 604/187

(57) ABSTRACT

To enable a user to readily determine the gauge of the needle of a needle assembly that has a base and a needle protective housing pivotably attached thereto, the needle assembly is injection molded from a color coded molding material which color was preassigned to correspond to the gauge of the needle. As a result, both the base and the protective housing of the needle assembly are the same specific color, and reflect or provide an indication of the given gauge of the needle. The needle sheath that covers the needle prior to use may be made of a plastics material that may be clear, or have the same or a different color than that of the needle assembly.

### FIELD OF THE INVENTION

The present invention relates to needle assemblies and specifically needle assemblies that have a protective housing connected to a base, with a color scheme for the needle assembly that relates to the gauge of the needle of the needle assembly.

### BACKGROUND OF THE INVENTION

In a medical environment, to draw blood from or inject fluid to a patient, in the case of a syringe being used, conventionally a needle is mated to the syringe by way of a hub to which the needle is attached or integrated. Depending on the procedure, the gauge of the needle tends to vary. It is well established that the gauge of the needle may be determined by the color of the hub. Yet the needle hub, or at least a portion thereof, is often hidden by the needle sheath that covers the needle when the needle is mated to the luer of the syringe.

With current technological and safety advances, a needle protective housing may be attached to the needle hub, with the needle hub, or base, and the needle protective housing being joined and either acting as an adapter as taught by U.S. Pat. No. 4,982,842, or as a single unit with the needle integrated to the base and the needle protective housing connected to the base as disclosed in U.S. Pat. No. 5,993,426 and U.S. Pat. No. RE37,110. The respective disclosures of the '842, '426 and '110 patents are incorporated by reference herein. In the devices disclosed by the aforenoted patents as well as in the prior art, there has not been any established scheme which allows a user to readily determine the gauge of the needle she is using. And given that the use of a needle with a particular gauge may well be of import in an emergency situation, there is a need to provide a scheme whereby a user can readily ascertain the gauge of the needle she would like to use.

### SUMMARY OF THE INVENTION

To enable a user to readily determine the needle gauge of a needle assembly that has a housing attached to a base or a needle hub to which the needle is attached, during the manufacturing process, color pigmentation that is specific to the gauge of the needle is added to the mold material, which most likely would be a plastic material such as polypropylene. With the mold material having the color that corresponds to the gauge of the needle in accordance with the ISO (International Standard Organization) standard, a user could readily pick out from among a plurality of needle assemblies the particular gauge of needle she wants to use. For example, a needle assembly with both the base and the housing being black would signify to the user that it is a 22 gauge (22 G) needle, while a blue needle assembly would indicate to the user a 23 G needle is in that blue needle assembly.

To provide a redundant or back up check for the user to determine the gauge of the needle for the needle assembly, the needle sheath that covers the needle prior to use may be made of clear plastic, so that the user can ascertain the gauge the needle visually, and confirm it with the color scheme of the needle assembly, or vis versa.

The needle device or needle assembly of the instant invention has a needle of a particular gauge and a base having attached thereto a needle protection housing that is pivotable relative to the base. Both the base and the needle protection housing are formed from a molding material that has added thereto a specific color pigmentation preassigned, pursuant to ISO standard, to a particular gauge of needle. With the needle of the particular gauge attached or integrated to the base or needle hub of the needle assembly, and given that both the base and the needle protection housing are of the same specific color, a user can readily ascertain the gauge of the needle by merely looking at the color of the needle assembly, i.e. either the base or the housing, or both. The needle protective housing is used to cover the needle once it is used, by being pivoted to a position in alignment substantially along the longitudinal axis of the needle assembly.

The instant invention also relates to the method of manufacturing the needle device that includes the steps of selecting a needle having a particular gauge, adding a specific color to a molding material, forming a needle assembly having a base and a needle protection housing pivotable relative to the base from the molding material so that the base and the housing have the same specific color, attaching the needle to the base, with the needle being coverable by the housing when the housing is pivoted to a position in alignment along the longitudinal axis of the base, and corresponding the specific color to the particular gauge of the needle so the user can ascertain the gauge of the needle by looking at the color of the needle assembly.

The instant invention also relates to the manufacture of a plurality of needle devices in which the respective colors are assigned to needles of different gauges. Moreover, different colored molding materials are provided and a color molding material with the color assigned to a given needle gauge is used for forming a plurality of needle assemblies each having a base and a needle protection housing pivotable relative to the base being the same specific color. Needles of the gauge that correspond to the specific color are attached to the base of the needle assemblies. When the job batch involving all of the needles of the same gauge have been attached to needle assemblies that have the color specific to the needle gauge, another job batch may begin whereby needles of a different gauge are attached to needle assemblies whose bases and needle protective housings have the same color that has been assigned to the needles with the different gauge. With the needle assemblies each being color coded to correspond to a particular needle gauge, a user can readily determine the gauge of the needle that she wants to use by merely looking at the color of the needle assembly.

### BRIEF DESCRIPTION OF THE FIGURES

The present invention will become apparent and the invention itself will be best understood by reference to the following description of the present invention taken in conjunction with the accompanying drawings wherein:

FIG. 1 is a perspective view of a needle assembly of the instant invention in which the base has attached thereto a needle protective housing;

FIG. 2 is a needle assembly of the instant invention that has attached to its base or needle hub a needle of a gauge that corresponds to the color scheme of the needle assembly; and

FIG. 3 is a flow chart illustrating an exemplar method of manufacturing the instant invention.

### DETAILED DESCRIPTION OF THE INVENTION

The present invention safety needle device comprises a needle assembly 2 that has a base 4 and a protective housing 6. Base 4 is connected to protective housing 6 by a living hinge 8. Base 4 has a needle hub 10 to which a needle 12 is attached at an opening 14. Once attached, needle 12 is sealed. Needle 12 could also be integrated to needle hub 10. Base 4 further has a luer end 16 which mates with a luer end of a syringe, as is well known in the art. Protective needle housing 6, for the embodiment shown, comprises a housing that has a longitudinal slot 18 through which needle 12 passes, when housing 6 is pivoted in the direction as shown by directional arrow 20 toward the longitudinal axis 22 of base 4 for enveloping or covering needle 12. A needle retention mechanism such as a hook 24 is provided in needle protective housing 6 for grasping needle 12, once housing 6 is pivoted to be in substantial alignment along the longitudinal axis 12. Once grasped by hook 24, needle 12 and housing 6 are retained relative to each other so that needle 12 is prevented from being further exposed. Note that instead of a hook, other needle retention mechanism such as the SIDE SNAP locking mechanism as disclosed in the afore-mentioned incorporated by reference U.S. Pat. No. RE 37,110 may also be used.

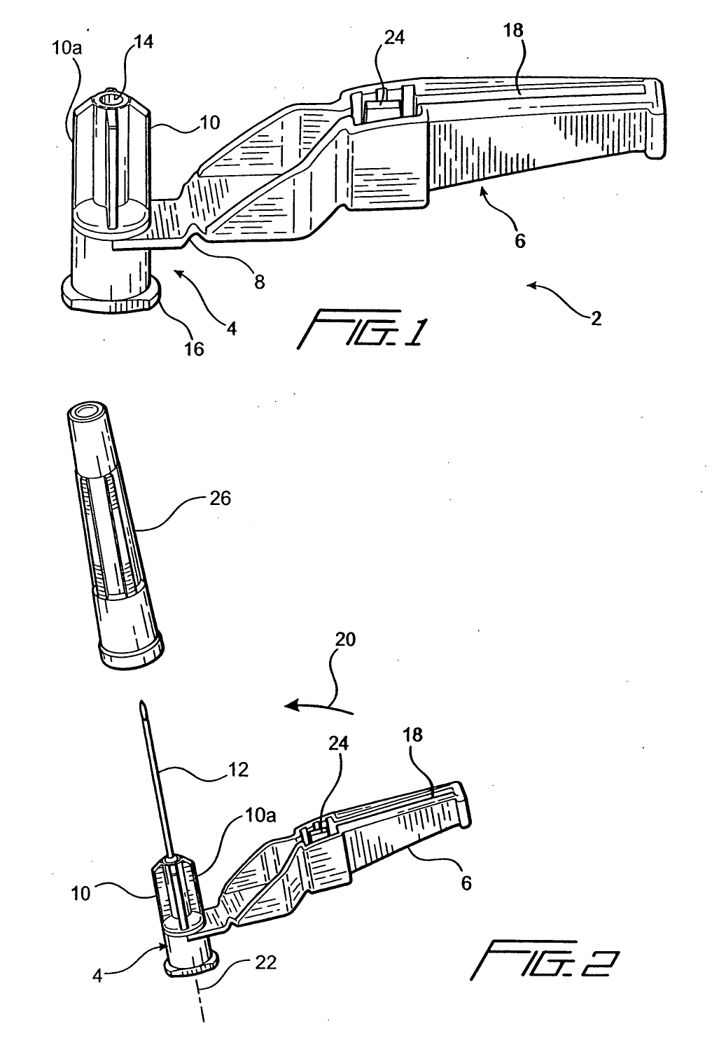

As best shown in FIG. 1, needle assembly 2 is a single unit that may well be injection molded from a medical plastics material such as for example polypropylene. In some instances, instead of being a single unitary unit, housing 6 and base 4 may well be molded as separate units with means provided at housing 6, such as for example a ring or a collar, that allows housing 6 to be attached to base 4. In any event, housing 6 and base 4 for the embodiment shown in the drawings are parts of the same needle assembly and are injection molded from a given conventional medical plastics molding material such as for example polypropylene.

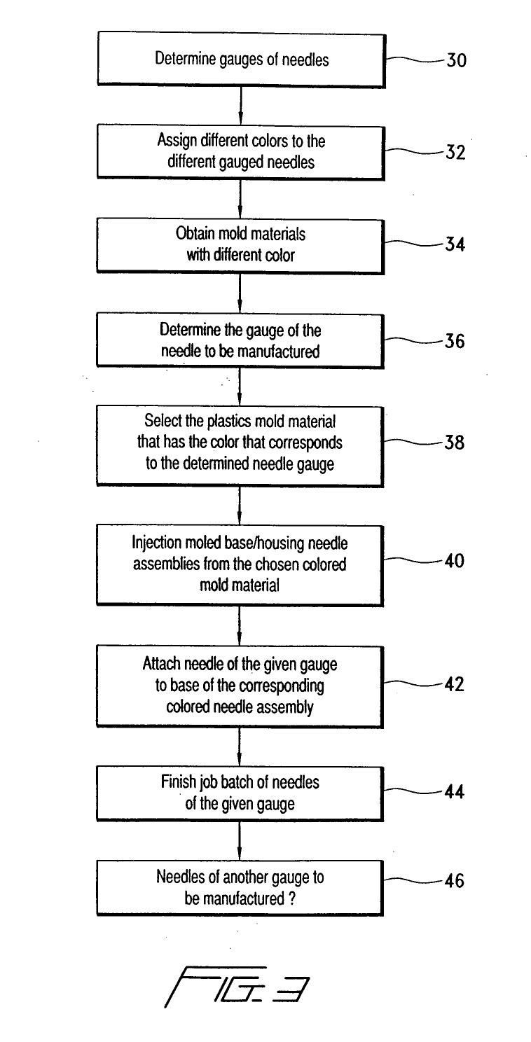

Because there are a number of different medical procedures, needle 12 may be manufactured with different dimensions, or different gauges, for example from 18 gauge to 30 gauge in conventional practice. Prior to the instant invention, a needle of a particular gauge is attached to a needle hub of a specific color. To use, the needle is mated to a luer end of a syringe. But the needle, whatever its gauge, is covered by a needle sheath such as component 26 shown in FIG. 2. Needle sheath 26 most likely is mated to needle hub 10 by frictional contact with the fins 10a that extend along the length of needle hub 10. Needle sheath 26 may well be made from an opaque plastics material so that once needle sheath 26 is fitted to needle hub 10 to cover needle 12, needle hub 10 may not be readily viewable. Indeed, insofar as the major portion of needle hub 10 is covered by the base of needle sheath 26, there is but a small portion of needle hub 10 that may be viewed by the user for determining the color of the needle hub. Accordingly, the gauge of needle 12 may not be readily ascertained.

The International Standard Organization (ISO) has assigned to the different gauged needles specific corresponding colors. Relying on the ISO standard, the inventors of the instant invention envision the color coding of the different gauged needles by making the complete needle assembly to which a needle is attached the specific color that is assigned to the particular gauge of the needle. In other words, assuming that a 22 gauge needle has been assigned the color “black”, that 22 gauge needle would be fitted or attached to a completely black needle assembly such as for example needle assembly 2 as shown in FIG. 1. The needle assembly may be injection molded from a molding material that has added thereto a black color pigmentation. Thus, the 22 gauge needle assembly is black at both its base 4 and its protective housing 6, as well as hinge 8. Similarly, for a 23 gauge needle, the needle assembly 2 to which the 22 gauge needle is attached would be blue, insofar as it is assumed that a 23 gauge needle has been assigned the color “blue”. For a 25 gauge needle, the needle assembly for that needle would be orange, pursuant to the “orange” color that has been assigned to a 25 gauge needle, etc.

With both the base 4 and the needle protective housing 6 being of the same specific color that corresponds to the gauge of the needle, a user can therefore readily determine from a glance of the needle assembly the gauge of its needle, even were the needle covered by a needle sheath 26 that is opaque. To provide a redundant means for a user to determine the gauge of the needle, needle sheath 26 may be made of a clear plastic material.

The method by which the gauge of a needle is color coded per the instant invention is discussed herein with the flow chart in FIG. 3. Assume that a plurality of needles of different gauges are to be manufactured. Further assume that there are a number of batches of needles to be manufactured, with the needles of each batch being of a particular gauge.

Starting at step 30, the process of the instant invention begins by determining the gauge, or gauges, of the needles to be manufactured. At step 32, different colors are assigned to the different gauged needles. At step 34, molding materials of different colors are obtained. Assume that for the manufacturing process, a batch of needles each having the same particular gauge are to be manufactured. That being the case, at step 36, the gauge of the batch of needles that are to be manufactured is determined. Thereafter, the molding material that has the color that corresponds to the determined gauge of the needle is selected, per step 38. With the appropriately colored molding material now selected, the process proceeds with the injection molding of the needle assembly, for the embodiment comprising both the base and the housing as a single unit, from the chosen colored molding material, per step 48. This injection molding process may be performed by a number of conventional injection molding machines such as for example the injection molding machines manufactured by the Nissei Company of Japan. With the molded needle assemblies on hand, the needles of the same particular gauge each are attached to the base, or the needle hub of the base, of one of the color coded needle assemblies, as shown in FIG. 2, per step 42. At the end of the manufacturing process after step 42, a needle assembly that has a needle of a particular gauge with a base and a housing both having the same specific color that corresponds to the gauge of the needle, such as that shown in FIG. 2, is produced. Thereafter, a determination is made per step 44 to see whether all of the needles of the same particular gauge have been assembled to corresponding needle assemblies for that job batch. And if it is determined that the job batch is finished, then the process proceeds to step 46 where it is further determined whether there are other needles of other particular gauges that need to be manufactured. If there are, then the process once again proceeds as outlined above starting at step 36. On the other hand, if there are no other color coded needle assemblies to be produced, then the manufacturing process is stopped.

It should be appreciated that even though the needle sheath as disclosed above is made of either an opaque material or a clear plastics material, it may well be that the needle sheath may also be made of a material that may have the same color as, or a different color than, that of the base and protective housing of the needle assembly. Furthermore, even though the needle assembly as shown in FIGS. 1 and 2 is formed as a unitary unit, it may well be that the base and the protective needle housing may be formed as separate units from different molds and be connected later during the manufacturing process. If the needle sheath were also molded from the same colored molding material as the base and the needle protection housing, the needle sheath that covers the needle prior to its use may therefore also be color specific to the particular gauge of the needle.

1-14. (canceled)

15. A needle device, comprising: a needle having a particular gauge, a needle assembly having a base and a needle protection housing pivotable relative to said base formed from a molding material, said molding material having added thereto a specific color pigmentation preassigned to correspond to said particular gauge, said base and said housing each having said specific color, said needle being attached to said base, said needle adapted to be covered by said housing when said housing is pivoted to a position in alignment along the longitudinal axis of said base, wherein the gauge of said needle can be ascertained by looking at the color of said needle assembly.

16. Needle device of claim 15, further comprising a needle sheath covering said needle to prevent contamination of said needle before use.

17. Needle device of claim 16, wherein said needle sheath has a different color than said base and housing.

18. Needle device of claim 16, wherein said needle sheath is formed from clear plastic so that a user can further ascertain the gauge of said needle without removing said needle sheath.

19. Needle device of claim 15, further comprising a needle retaining mechanism in said housing for preventing relative movement between said needle and said housing after said housing has been pivoted to said alignment position to cover said needle.

20. A needle assembly having a base and a needle protection housing pivotable relative to said base, said base and said housing formed from the same molding material, said base having a needle that makes contact with a patient, said needle adapted to be covered by said housing when said housing is pivoted to a position in alignment along the longitudinal axis of said base, said needle having a particular gauge, said molding material having a specific color that corresponds to said particular gauge of said needle according to a given standard so that the gauge of said needle corresponds to the color of said needle assembly.

21. Needle assembly of claim 20, wherein said molding material is plastic; and wherein said base and said housing are injection molded as a one piece needle assembly.

22. Needle assembly claim 20, further comprising a needle sheath for covering said needle to prevent contamination of said needle before use.

23. Needle assembly of claim 22, wherein said needle sheath has a different color than said base and housing.

24. Needle assembly of claim 22, wherein said needle sheath is formed from clear plastic so that the gauge of said needle is viewable without removing said needle sheath.

25. Needle assembly of claim 20, wherein a needle retaining mechanism is formed in said housing for preventing relative movement between said needle and said housing after said housing has been pivoted to said alignment position to cover said needle.

26. Needle assembly of claim 20, wherein said base and said housing are molded as attachable separate pieces having the same color from said molding material.

27. Needle assembly of claim 20, wherein said base is matable to a syringe.

28. A one piece needle safety device, comprising:
a needle assembly having a base and a needle protection housing pivotable relative to said base;
a needle for use in drawing or injecting fluid to a patient extending from said base, said needle having a given gauge, said needle covered by said housing when said housing is pivoted to a position in alignment along the longitudinal axis of said base;
wherein said base and said housing are formed as a unitary piece from a molding material that has a specific color that corresponds to the given gauge in accordance to a standard so that the given gauge of said needle is represented by the specific color of said base and said housing.

29. Safety device of claim 28, further comprising a needle sheath that covers said needle to prevent contamination of said needle before use.

30. Safety device of claim 29, wherein said needle sheath is transparent or has a different color than said base and housing.

31. Safety device of claim 28, wherein said base is matable to a syringe.

32. Safety device of claim 28, further comprising a catch mechanism at said housing that prevents relative movement between said base and said housing once said housing is pivoted to cover said needle.
